(19) United States
(12) Patent Application Publication
(10) Pub. No.: US 2003/0050555 A1
(43) Pub. Date: Mar. 13, 2003

(54) MR injector system with increased mobility and electromagnetic interference mitigation

(76) Inventors: Richard G. Critchlow, Oakmont, PA (US); Charles J. Mutschler, Oakmont, PA (US); John D. Shearer JR., Herminie, PA (US); John R. Gardner, Wexford, PA (US); Joelle A. Rudnick, Moon Township, PA (US); Timothy J. Maiese, Cranberry Township, PA (US); Dennis R. Presky, Washington, PA (US); Michael V. Pogozelec, Cranberry Township, PA (US); John F. Rihn, Glenshaw, PA (US)

Correspondence Address: GREGORY L BRADLEY, MEDRAD INC, ONE MEDRAD DRIVE, INDIANOLA, PA, 15051

(21) Appl. No.: 10/116,765

(22) Filed: Apr. 3, 2002

Related U.S. Application Data
(60) Provisional application No. 60/281,042, filed on Apr. 3, 2001.

(51) Int. Cl. A61B 5/05

(52) U.S. Cl. 600/420

(57) ABSTRACT

An improved magnetic resonance imaging (MRI) injection system exhibits decreased electromagnetic interference (EMI) and improved mobility. The injector apparatus of the injection system includes a head assembly with at least one drive piston and a base assembly including the motor control circuitry and drive motors necessary to control the injector. The electronics in the base assembly are designed to reduce EMI with the magnetic field of the MRI system by employing Faraday cages and various circuit design and filtering techniques. The head assembly of the injector apparatus may also include a hand switch for local control of the injector.

### CROSS-REFERENCE TO RELATED APPLICATION

This application claims the benefit of U.S. Provisional Application Serial No. 60/281,042, filed on Apr. 3, 2001, the contents of which are incorporated herein by reference.

### BACKGROUND OF THE INVENTION

This invention relates generally to the field of Magnetic Resonance Imaging (MRI) systems for generating diagnostic images of a patient's internal organs. More particularly, this invention relates to improved MRI contrast media injection systems exhibiting decreased electromagnetic interference and improved mobility.

MRI systems require isolation from external sources of electromagnetic fields if optimum image quality is to be obtained from MRI diagnostic procedures. Conventional MRI systems typically employ some form of electromagnetic isolation chamber (i.e., a “Faraday cage”) which is typically a room enclosed by copper sheeting or conductive mesh material that isolates the interior of the scan room from undesirable sources of electromagnetic radiation and the electromagnetic noise inherent in the atmosphere.

However, not all components of or used with an MR system can be placed outside of the protective shield of the scanning room. For example, a contrast media injection system, which is used to administer a contrast agent into the patient's body to enhance MR images, must be located adjacent to the patient. Because the MR scanner is designed to capture and interpret RF (radio frequency) energy, the electronics and other components of the injection system, which emit RF energy, may disrupt the MR system.

Specifically, the scanning room inherently includes a “noise floor” of RF energy that always is present in the scanning room (radiating from the lights, heating system and other existing devices). The MR scanner is designed to account for this noise floor when constructing an MR image. However, any additional RF noise (above this floor) may be detected by the MR scanner and appear in the MR image as an artifact (a false, non-patient-generated feature in the MR image).

Generally, the scan room-based injector system imparts two types of interference to the MR suite. First, all of the ferrous (magnetic) material introduced into the scanning room (e.g., from the enclosures and electronics of the injector system) distorts the MR scanner-generated magnetic field creating a homogeneity error, which can result in a geometric distortion in the MR image. Additionally, any motors or electronics in the injector may emit electromagnetic interference (EMI) that causes RF artifacts (e.g., a horizontal or vertical line in the MR image) to appear on the MR image.

Attempting to reduce these artifacts by adding electronic filters (to reduce EMI) may introduce further complications into the design of an injection system. For example, an inductor-based filter will easily saturate when placed within the intense magnetic field of an MR scanning room. This may cause the “actual” inductance of the inductor to be much lower than expected.

As a general rule, the motors that control the syringes in the injector head are preferably located as close to the syringes as possible. However, direct connection of typical motors is impossible because of the magnetic attraction of the motors to the bore of the magnet, the adverse effect of the motor operation on the MR image, and the adverse effect of the magnetic field of the MRI system on motor operation. Therefore, improved motor design, location and/or orientation may improve the effectiveness of the injection system.

A conventional motor control arrangement for an MRI injection system is disclosed in U.S. Pat. No. 5,494,036, the disclosure of which is incorporated by reference. As disclosed in the '036 patent, remotely-located motors of the injection system are connected by flexible drive shafts to drive pistons in an injector head, which is located adjacent to a patent to be imaged by the MRI scanner. The drive pistons, in turn, are connected to and drive plungers in syringes that are releasably engaged with the injector head. (A suitable arrangement for attaching a syringe to an injector is described in U.S. Pat. No. 5,383,858, the disclosure of which is hereby incorporated by reference.) As the flexible shafts rotate, the drive pistons cause linear movement of the plungers in the syringes to inject contrast media into the patient.

However, conventional MRI injection systems, such as the one described above, are still not ideal because of the additional electromagnetic interference of the remote motors and the still unsatisfactory mobility of the scan room injector unit.

Accordingly, there is a need to provide an improved magnetic resonance imaging contrast media delivery system (scan room unit) having decreased interference between the electromagnetic field used to obtain the magnetic resonance image and the electromagnetic fields created by the injection equipment. This system preferably provides an injection apparatus with decreased electromagnetic interference emitted from the electronics and electric motors used to drive the contrast media injection system. The injection system may also maintain MR scanner field homogeneity better than prior systems. The injection system is also preferably more mobile than prior injection systems.

These various limitations to the current implementation of an MR injection system are preferably improved in relation to the prior art through the use of the current invention.

### SUMMARY OF THE INVENTION

In accordance with the invention, there is provided a system for an improved MR injection system with a decreased amount of electromagnetic interference between the magnetic resonance imaging (MRI) system and the injector system. This MR injection system preferably includes a scan room system arrangement that allows greater mobility of the injectors.

In a preferred embodiment, the invention provides an injection system for use with an MRI system. The MRI system has a room and a window therewith shielded from electromagnetic interference. The injection system comprises a system controller external to the room, an injection apparatus within the room, and a communications control link that permits communication therebetween. The injection apparatus includes a base assembly, a head assembly, and a rigid tubular casing for supporting the head assembly above the base assembly. The tubular casing also houses at least one flexible drive shaft. The base assembly includes motor control circuitry and at least one electric drive motor controlled thereby. The electric drive motor is operably linked to one end of the flexible drive shaft. The head assembly includes an injector and at least one drive mechanism therefor. The drive mechanism is operably linked to the other end of the flexible drive shaft. The communications control link enables the system controller to operably control the base assembly and the head assembly therewith and the internal components therein and therebetween such as the flexible drive shaft(s) and the drive mechanism(s). It ultimately controls operation of the drive mechanism(s) of the injector and the at least one syringe mounted thereon through which fluid(s) can be injected into a patient during a magnetic resonance imaging procedure.

In a related aspect, the invention also provides a roll-resistant caster assembly for an injection apparatus of the type used with a magnetic resonance imaging (MRI) system. The caster assembly comprises an axle, two wheels, a housing, a dual disc pad element, and a spring. Each wheel defines a hub for attachment to one end of the axle, and also defines a drum on an interior periphery thereof. The housing has a fork portion characterized by two prongs projecting downwardly therefrom. Each prong defines an axle bore therethrough. The axle bores of the prongs are axially aligned to accommodate the axle for support of the housing thereon. The housing also has an upper spring stop situated between the prongs in an upper part of the fork portion. The dual disc pad element has two discs and a connective member interconnecting them so that they are oriented parallel to each other. The discs each define a central bore to accommodate compressive movement of the dual disc pad element relative to the axle situated therein and the fork portion therewith. The connective member includes a lower spring stop at a top and a center thereof. The connective member also defines two slots each of which on opposite sides of the lower spring stop and each adapted to accommodate one of the prongs of the fork portion of the housing. The spring is disposed compressively between the upper and lower springs stops such that it normally biases the discs of the dual disc pad element against the drums of the wheels thereby rendering the wheels of the caster assembly resistant to rolling.

The present invention is not limited to those examples discussed above. These and other objectives and advantages of the present invention will become readily apparent to persons skilled in the art from the following description of the particularly preferred embodiments of the invention.

### BRIEF DESCRIPTION OF THE DRAWINGS

The invention and its presently preferred embodiments will be better understood by reference to the detailed disclosure below and to the accompanying drawings, wherein:

FIG. 1 is a block diagram of the MR injection system of the present invention;

FIG. 2 is an isometric view of a scan room unit (injection apparatus) of the present invention;

FIG. 3 is a block diagram of the base assembly components of the present invention;

FIG. 4 is a block diagram of the power drive card of the present invention;

FIG. 5A is a perspective view of a roll-resistant caster according to a preferred embodiment of the invention;

FIG. 5B is an exploded view of the roll-resistant caster shown in FIG. 5A;

FIG. 5C illustrates side and front (back) views of a dual disc friction pad element of the roll-resistant caster shown in FIG. 5A; and

FIG. 5D illustrates a perspective view of the roll-resistant caster of FIG. 5A with one end cap removed to show the flanged end of the axle.

### DESCRIPTION OF THE PREFERRED EMBODIMENTS

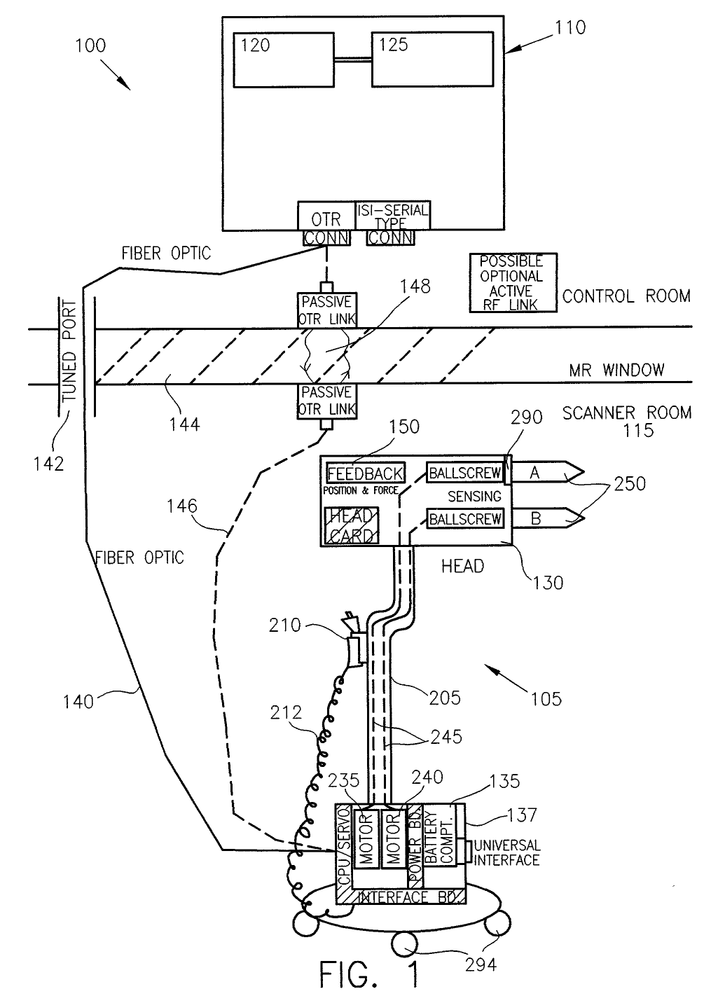

In at least one preferred embodiment, the present invention comprises an improved MR injection system including a programmable multiple (e.g., dual) syringe system designed to administer controlled doses of intravenous MR contrast agents and common flushing solutions to patients undergoing a contrast-enhanced MR scan. As seen in the FIG. 1 block diagram of an MR injection system 100, the two basic components that make up the MR injection system 100 include a scan room unit 105 and a control room unit 110. The scan room unit (or contrast agent injection apparatus) 105 is typically located within the electro-magnetically shielded (Faraday cage) scanning room 115 in which a patient undergoes an MRI. The control room unit 110 controls the injection process from outside the scan room 115 (to reduce EMI in the scan room).

As a preliminary matter, it should be noted that when used herein to describe the inside of the scanning room 115 during an MR scan generally, the terms “electromagnetic” and/or “EMI” refer to both the RF and magnetic fields and radiation. However, when used to describe the effects on a specific component, such as an inductor, the term “electromagnetic” refers to the magnetic field only—which saturates the inductor—because the RF component does not change the inductor's performance.

The control room unit 110 preferably houses a touch screen or other display 120 as well as electronic components 125 used to program the injection apparatus 105. The injection apparatus 105, which may be positioned near the magnet bore, preferably includes an injector head 130, a battery pack 135, and the mechanical and electrical assemblies required for fluid (contrast agent) delivery to the patient. These two devices 105, 110 are generally in communicative contact via a fiber optic link or other low-noise communications channel 140.

Because of the intense and changing electromagnetic fields within the MR scanning room 115, the injection apparatus 105 is susceptible to a large amount of field interaction. Likewise, the sensitive receivers in the MRI suite are susceptible to any RF noise emitted from the injection apparatus 115 with an amplitude greater than the “noise floor.” Specifically, any electronics, motors, or other devices in the injection apparatus 105 that emit RF interference may cause artifacts to appear in the MR image and any metal in the scanning room may alter the homogeneity of the MRI electromagnetic field. Conventional filtering solutions to these problems may be made more difficult because of the inductor saturating effects of the intense electromagnetic field in the scanning room.

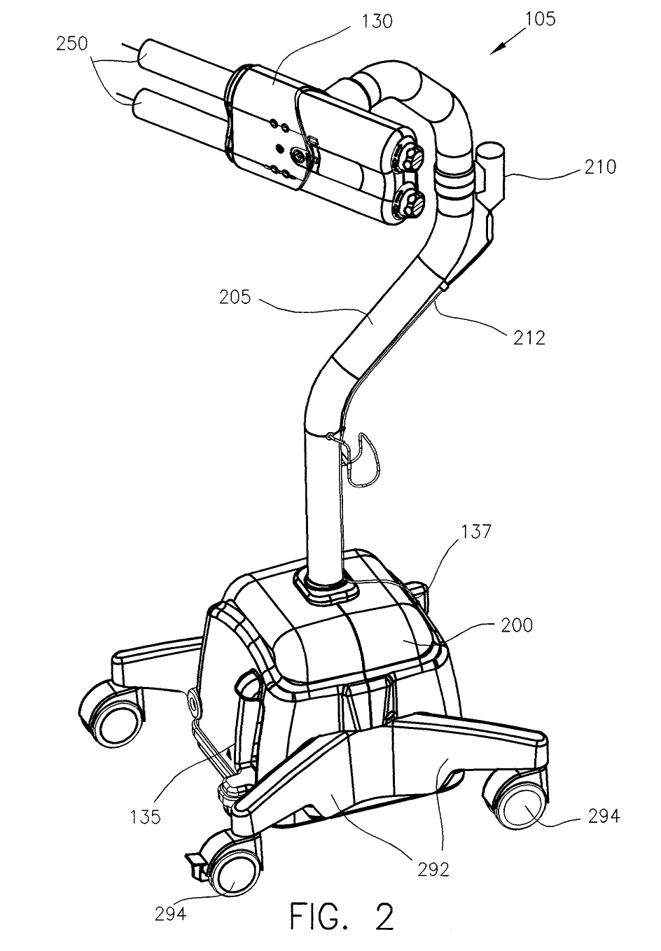

As best shown in the FIG. 2 isometric representation of one embodiment of the injection apparatus 105, the injection apparatus preferably includes an injector head 130, a lower console (or “base assembly”) 200 and a hollow neck or tubular casing 205 connecting the base assembly 200 to the injector head 130. The injection apparatus 105 may also include a hand switch 210 for local control of the injection system and a removable system battery pack 135 in the base assembly 200, which provides power to the motors, and electronics of the injection apparatus 105.

The injector head 130 preferably includes a plurality of components according to FIG. 1. For example, there may be a sensor/feedback card 150 that receives feedback from the absolute positioning devices indicating the linear positioning of the drive pistons (e.g., relating to the amount of contrast that has been injected into the patient). There may also be a switch card that interfaces with lighted indicators that indicate the state of the injector.

The injector head sensor card 150, which may be connected to a bridge assembly, receives a differential signal from the injectors (indicating force) and sends this information back down to the base (via the interface card) for processing. This feedback may be important to ensure and correct for proper functioning of the drive pistons.

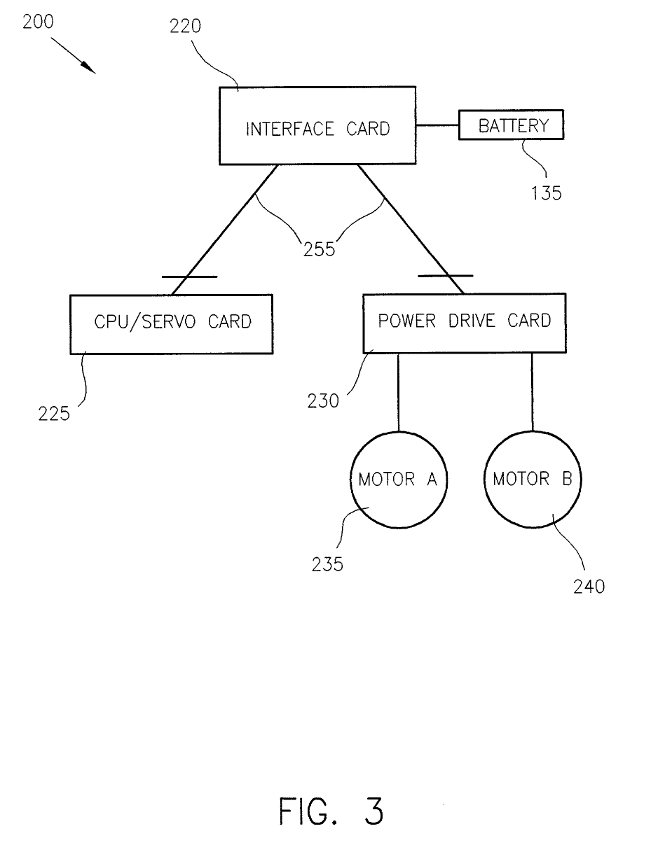

The base assembly 200 preferably includes a plurality of circuit boards (e.g., CPU/servo card 225, interface card 220 and power drive card 230 and motors 235, 240 which provide functionality to the injector head system as detailed in the block diagram of the base assembly in FIG. 3. The power drive card 230 transfers power to a plurality of motors 235, 240 that drive flexible shafts 245 that lead up the rigid neck 205 of the scan room unit 105 into the injector head 130. These flexible shafts 245 convert the rotational motion of the motor shaft to linear motion of the drive pistons. In the injector head 130, the flexible shafts 245 are in mechanical contact with a drive piston capable of forcing a liquid medium (such as an MR contrast agent or flushing solution) out of a syringe 250 and into a patient. In a preferred embodiment, there are two detachable syringes 250 attached to two pistons and two corresponding motors 235, 240 within the base assembly.

The power drive card 230 is also in electronic communicative contact with an interface board 220 that is connected to both a power supply (battery) 135 and a CPU/servo controller board 225. The power drive card 230 converts low level PWM voltage signals from the CPU/servo card 225 to current signals at the output of the power amplifier that are proportional to the PWM duty cycle. The interface card 220 generally acts as an interconnection point and communications link between the various boards in the base assembly 200 and devices of the control room injection unit.

The boards in the base assembly transmit and receive information over a conventional data bus 255. In the prior art systems, when the motors turn on, there is a large current rush into the motors to satisfy the need for power. This causes the voltage on the 12 VDC to drop down sharply for a brief period of time. Because of the PWM of the motors, this voltage drop is repeated at about 16 kHz.

The capacitance of the bus lines 255 may be increased to decrease the voltage drop in these lines when the motors 235, 240 turn on. In a preferred example embodiment, if the capacitance of the bus lines 255 is increased from 100 μF up to about 330 μF, this voltage drop is decreased and substantially eliminated without substantially affecting the current draw of the motors 235, 240.

The bus capacitance of the interface card 220 may be increased by inserting a capacitor in parallel with the bus lines fed back to the interface card. As just described, a capacitor of approximately 330 μF has been shown to be appropriate, but this capacitor value should be taken into account when determining the proper resistor value for the RC time constant on each voltage leg of each motor (described in more detail below).

The base assembly 200 also preferably includes an interface (see FIG. 1) that may transmit and receive signals to and from the control room unit 110. Typically, this interface will be by way of a fiber optic cable 140 which passes through a tuned port 142 in the wall 144 between the scan room 115 and the control room and provides a communicative link with little or no incoming RF interference. Alternatively, an optical transceiver link 146 maybe established from the scan room 115 to the control room through a window 148 in the scan room 115. In addition, a wireless RF link (which operates outside of the frequency range of the MRI system) could be employed as the interface. Suitable optical transceiver and wireless RF links are described in U.S. Pat. No. 5,494,036 and U.S. application Ser. No. 09/586,140, filed on Jun. 2, 2000, the disclosures of which are hereby incorporated by reference. However, any suitable communications medium that emits low amounts of RF interference may be employed.

The various component parts and interconnections within the base assembly 200 typically emit electromagnetic interference, which may impair or distort the quality of the digital image created by the MRI scanner. The noise from each of these distinct components may be communicated to other components, or may be radiated directly out of the base assembly enclosure 137. Specifically, each board 220, 225, 230 may radiate EMI, the noise may couple to the base assembly enclosure 137 and manifest itself in the scan room 115. Some noise may travel up into the injector head 130 where it can be directly received by the scanner receivers in and around the MRI bore.

For example, the CPU/servo board 225 may radiate various frequencies of electromagnetic noise emanating from the digital signal processing. This may include both spurious noise from the conductive lengths etched on the board, as well as some EMI radiating from the component parts (such as the microprocessors). This digital noise may be propagated to the interface card 220 or out of the enclosure around the base assembly.

Likewise, the power drive card 230 may produce spurious noise (switching noise) that may be propagated up to the interface card 220 (and out of the base assembly enclosure 137). Again, this noise is preferably filtered out before it is propagated to the interface board 220.

The base components are preferably encased in an enclosure 137 that operates as a Faraday cage around the internal components. As is common in the art, a Faraday cage (at least theoretically), prevents an electric charge outside of the cage from penetrating the interior regions of the cage or conductive shell. The more complete the conductive shell, the better the charge insulation becomes. Therefore, the base housing 137 may be designed to form an almost continuous conductor around the base assembly components.

For example, the base enclosure 137 may be formed from a metal extrusion process to reduce the number of seams in the enclosure. Likewise, the distance between any two screw holes, which may act as slotted antennas, is designed and fabricated so that these unwanted antennas won't transmit electromagnetic frequencies generated by the MRI system into the enclosure 137, and won't transmit EMI from inside the enclosure 137 out to the rest of the scanning room 115.

The enclosure 137 is typically made of a conductive but non-ferrous material, such as bronze, and is coated with an additional layer of conductive materials (such as gold chromate). Any gaskets that exist, for example where external wires connect to the enclosure are preferably designed to inhibit EMI penetration. These external communications wires (e.g., out to the injector head), are preferably also shielded to “expand” the Faraday conductive cage to include all of the components connected to the base enclosure 137.

The injector head 130, if required, may also be an extension of a Faraday cage of the base assembly enclosure 137 (described above). This may be accomplished by coating the head 130 with a conductive material, creating a metallic enclosure or another means of continuing the shield from the base-to-head cable. Once again, this shield is tied to battery ground, via the cable or otherwise.

The injector of the present invention preferably uses a plurality of three-phase brushless motors 235, 240 to control the injection of contrast into the patient. Existing injector systems have heretofore utilized conventional DC brushed, piezoelectric or some other type of motor. These traditional motors are not ideally suited for use in the MRI environment.

For example, one prior art motor was a conventional three-phase brushed motor. Although this motor produced the required torque with a nominal sized battery, this motor radiated an appreciable amount of electromagnetic interference and also suffered from the altering affects of the MRI magnetic field on the motor operation (i.e., non-linear response of the motor to the electromagnetic field). Therefore, these motors were typically located at least 10-15 feet from the injector head (away from the strongest part of the MRI field). This remote location necessitated the use of a fairly long flexible transmission cable (to transfer the motor rotation to the syringe pistons) and an additional communication wire (from the motors to the injector head) causing both a dangerous situation within the scan room (e.g., tripping hazard) as well as providing another potential source of RF interference. See U.S. Pat. No. 5,494,036, the disclosure of which is hereby incorporated by reference.

Generally speaking, when using a three-phase brushless motor in a magnetic field, the current (required for rotation) in each leg of the motor windings increases proportionally to the influence of the external magnetic flux. Through testing, the effects of the external magnetic field on the motor's performance can be predicted and compensated for in system design.

The orientation of the motors 235, 240 in the base assembly 200 may also affect motor performance. For example, the motor positioning can be oriented to minimize the torque needed to move the flexible shafts 245 and therefore the injector's syringes 250. Testing shows that if the motors 235, 240 are placed offset between approximately 10 and 30 degrees from vertical, the needed torque may be minimal.

Also, any MRI field effects on the motor performance may be limited by orientating the electric motors 235, 240 in the base assembly 200 in a way that limits the flux through the motor coils. Specifically, the orientation should minimize the amount of MRI magnetic field lines that pass through the three-phase motor coils. At the location of motor placement, the magnetic field is generally vertical. By positioning the motors 235, 240 in a generally vertical direction, aligned with the magnetic field produced by the MRI system, the amount of the MRI-produced electromagnetic field that passes through the motor coils, and hence the amount of extra current drawn in the legs of the motor, may be decreased. With this orientation, the injector apparatus 105 can take advantage of the high-torque and low RF radiation of the three-phase brushless motor 235, 240, while drawing a reduced amount of current from the battery 135 (allowing a smaller battery to be used in the injector system and/or extending the life of the battery).

The brushless motor design, with improved orientation, makes the system, “quieter,” more mobile, and safer than the prior art system. Due to the behavior of the brushless motors 235, 240 in the MR environment, the present injectors may be positioned closer to the MR magnet (i.e., in the base of the injection apparatus) than prior injection systems. Attaching the motors 235, 240 and electronics to the same frame as the injector head may also increase system mobility over prior injection systems.

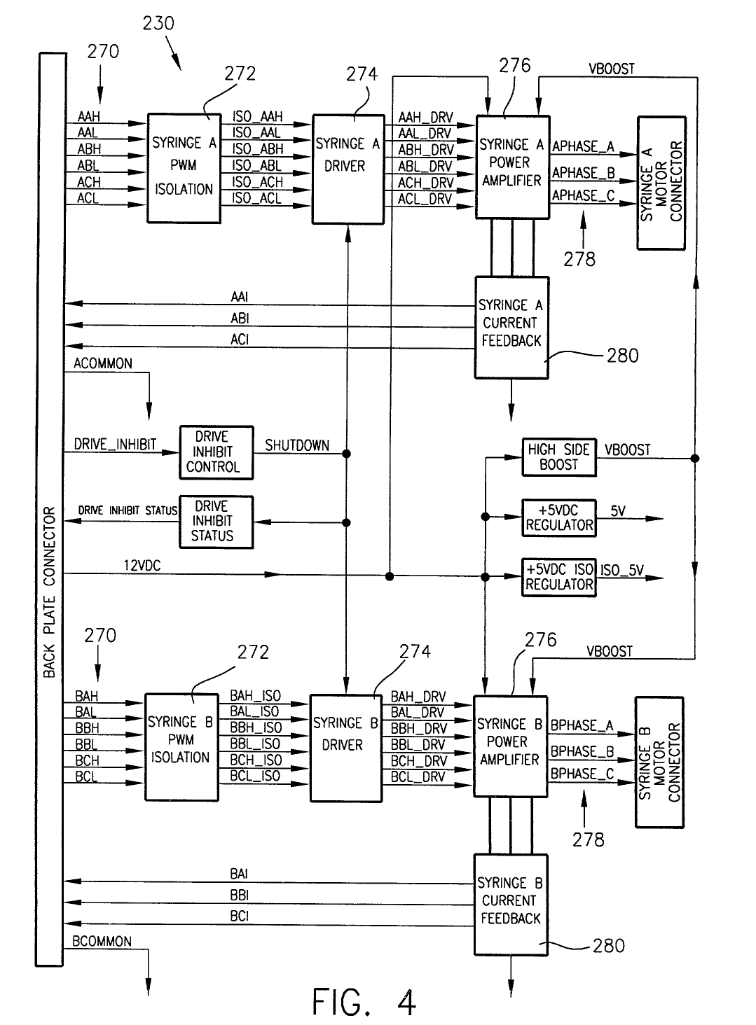

The motor power drive board 230 is shown in block diagram form in FIG. 4. As seen in FIG. 4, there are two sets of incoming three-phase signal voltages 270 to control each of the two motors 235, 240 that control the two syringes 250 in the injector head 130. The timing of these signals 270, as applied to the motors, is typically controlled by components on the CPU/servo card 225 and is passed to the power drive card 230 through the interface card 220. The PWM signals are initially isolated from the amplifier stage by passing the signals through optocouplers 272 for each motor.

The driven outputs of the syringe driver circuit 274 are fed into power amplifiers 276 for each of the PWM signals for each leg of the three-phase motors. The output of this power amplifier provides the three-phase voltage 278 to the two brushless motors that control the dosage of contrast fluid injected into the patient. These syringe motor power amplifiers 276 also output a current feedback 280 for each of the legs of the motor power source. This current feedback 280, sent back through the interface card 220 to the CPU/servo card 225, allows the injector system to determine when too much current is being drawn by a motor and to take the appropriate response action.

The switching involved to provide the proper three-phase power to the two syringe motors utilizes a conventional Field Effect Transistor (FET) H-bridge configuration (typically 6 MOSFETs—one on each leg of each motor) that are switched between providing PWM signals to each of the three power legs of the motor. This switching changes the magnetic polarity of the windings of the brushless motor and causes the motor's shaft to rotate in response to the changing magnetic field.

The power amplifier output, however, contains noise that may be propagated directly to the interface card via the bus or common lines. The attenuation of this noise is complicated by the use of an inductor in the MRI system's electromagnetic field (because of inductor saturation). Also, because the various voltages passed to the motor must change quickly (to cause the shaft to rotate quickly), any filtering must not slow down the rise time of the voltage on any of the motor power legs. Preferably, this noise is filtered both on the power drive board, as well as between the power drive board and the interface board or the motors.

The power FETs used in each amplifier require the use of gate-driven resistors. The resistors limit the in-rush current as seen by the output of the driver circuitry. These resistors, in conjunction with the intrinsic gate to source capacitance, form an RC network. By adjusting the gate resistance, one can alter the turn-on time of the FETs. This results in rapid switching transit times, which also results in high EMI radiation. Increasing the resistance, R, slows transit turn-on times at a cost of increasing power dissipation within the device. Therefore, R must be chosen to optimize either situation.

If the RC time constant is too high, the switching will be “hard” causing high frequency noise generation that may then be radiated (as noted previously) throughout the system, and eventually to the scanner receivers. It has been shown that a resistor value of approximately 100 Ohms matched with a capacitor on the bus lines of 330 μF (discussed previously) may have a steep enough rise time with sufficiently low EMI radiation.

To attenuate the noise emanating out of the power drive board 230 to the interface board 220, there is preferably inserted an inductor in-line with the bridge bus voltage transferred back to the interface card. These inductors are designed to filter the noise from the bridge bus voltage.

However, because the injector system operates within an intense electromagnetic field, the inductor will not operate according to traditional notions of circuit theory. Typically, the inductor will become saturated in the electromagnetic field and the inductance value will decrease. Therefore, testing is preferably performed to indicate how the inductor is affected by the MRI field, relative to the location of the subsystem to the scanner magnet, and the change in the inductance value is noted. Thereafter, a new value for the inductor (which takes into account the effects of the magnet), can be computed and implemented. For example, in an 800 Gauss magnetic field, an inductor value of approximately 100 μH may be appropriate to electro-magnetically insulate the power card from the interface card. Several different versions of the injection assembly may be produced with different inductor values to be used in magnetic fields of different strengths. Alternatively, some type of variable or selectable inductor may be employed for system flexibility.

In addition to the filtering steps described above, the layout of the circuit planes that make up the power drive 230, the interface 220 and/or the CPU/servo 225 cards are also preferably optimized to reduce electromagnetic radiation. For example, the layers of component parts are preferably ordered to minimize EMI. Specifically, the power circuits, signal circuits, ground planes, and other signal types should all be isolated from each other in a layered orientation that isolates analog from digital signals, data signals from power traces, power traces from ground planes, and so forth.

The ordering of the layers of the board in this “sandwich-style” configuration may be partially dictated by external factors. For example, the traces of inner layers that carry larger currents typically need to have much wider traces than inner low current traces or outer layer high current traces. Therefore, these higher current traces usually are placed on the outer layers of the board stack. In the power drive board 230, the current feedback and motor voltage lines may be located on the outer layers. However, the inner layers are preferably matched to minimize the amount of radiation. This orientation does not minimize EMI, but may be necessary for fabrication criteria.

Just inside the first “high current” outer plane is preferably a primary ground plane. The next layer may be a first signaling plane that contains components that carry some type of signaling voltage. After this first signal plane may be a power plane and then another ground plane (preferably the isolated power supply ground plane). Below these planes, a second signaling plane preferably exists, which may be followed by a final ground plane and the second high current plane on the outside opposite the first outer high current plane. With this layered configuration, each signal-carrying plane is immediately adjacent to a ground plane through which any noise can be sunk. The capacitive effects of the layer reduce the noise and minimize the trace lengths of “return” traces that seek a ground connection.

In addition to channeling the noise directly into an adjacent ground plane, each ground plane is preferably connected together and then connected to the ground on the battery. This configuration preferably sinks the noise into the battery (which is itself a large capacitor) and prevents most of the noise from escaping the power drive board 230.

Finally, the components on each of the layers of the board are preferably designed and fabricated to minimize the distance between components that interact with each other. By reducing the “trace” between the components, there is an increased amount of inductance that may create current loops that will radiate EMI. Also, the current flow through the components of each layer should be fabricated in “hook-shaped” or other non-closed configuration to minimize the possibility that a current loop or well may exist on the board. All of these layout measures preferably limit the electromagnetic radiation of the parts of the injector system.

As an ancillary benefit to the “non-looped” component layout, troubleshooting for EMI leaks on each system board may be facilitated. For example, if the components on a board plane are arranged to have large current loops, the board may “glow” or generally radiate with a large amount of detectable electromagnetic radiation. This glow inhibits the ability for a troubleshooter to find “hot spots” or particular areas of local EMI radiation that may be addressed. Once the “glow” is reduced, these hot spots are uncovered and the appropriate area can be individually addressed to further reduce RF interference in the system.

The interface card 220 is also preferably designed to reduce EMI. The layering of the interface card component planes may alternate between ground planes and signals planes and power planes to again effectuate a capacitive coupling between adjacent planes that shortens the lengths of current runs between the boards. Additionally, the components on each board are preferably located so that components that interact with each other are located near each other on the board (short traces) and minimize the number of current loops for signals traveling on the board. This is similar to the power drive card described above.

The general architecture of the board planes just described sinks any noise to the battery 135 (which is itself a large capacitor) and limits the amount of noise that radiates out of the base enclosure to eliminate or lessen artifacts in the MR image. In general, the board planes are laid out so that signal planes are adjacent to ground planes which sink the noise to the ground plane. These ground planes are then preferably connected to each other and to the battery ground, thus sinking the noise to the battery 135. Then, the Faraday cage enclosure 137 around all of the components in the base shields any noise that is not properly sunk to the battery 135. This enclosure 137 is also preferably connected to the battery ground. In this way, multiple cross-related technical solutions are utilized to reduce EMI from interfering with the MRI, even if the injector assembly is near the patient.

The layers of the cards in the base assembly 200 also act as Faraday cages by the selective use of ground planes to enclose any planes connecting electric signals. The base enclosure 137 acts as a second Faraday cage around the boards within the base assembly. Finally, the MRI chamber itself is typically designed as a Faraday cage (with a conductive shield built continuously around the room) that limits the amount of EMI from the outside world penetrating the system.

As described above and shown in FIG. 2, There may also be a hand switch 210 attached to the injector assembly 105 to program or select certain features of the injector system 100 from a location on or near the injector assembly 105. For example, the hand switch 210 may be removably attached to the neck 205 of the injector assembly 105. Preferably, the switch 210 allows an MRI operator to arm and disarm the injector (turn the contrast injection on and off) as well as pause the injector. Traditional MR injection systems do not allow this “pause” functionality, and any operator control from the injection apparatus typically consisted of a “start” and an “emergency stop” from which the injection process could not be easily restarted from the same point in the process.

The hand switch communications cable 212 extends from the base 200 of the injector (housing the injector motors). Because the hand switch 210 is in the MRI chamber 115, the hand switch cable 212 is preferably shielded from electromagnetic interference (EMI). This shield may take the form of a spiral braid that is helically coiled around the communications cable and electrically tied to the ground via the enclosure, directly to the battery 135, or in some other way. This type of shielding effectively expands the Faraday cage of the base assembly enclosure 137 and provides EMI protection while maintaining flexibility of the cable.

Because the communications medium 212 between the injector base assembly 200 and the injector head 130 may include RF noise originating in the base, the base-neck juncture may also include a PI filter to limit the amount of RF noise reaching the injector head 130. Because the injector head 130 is located near the patient, any RF interference is most likely to hinder the MRI image of the patient at this location. Therefore, this noise must be limited.

Preferably, the PI filter is a general PI filter, consisting of an inductor connected between two capacitors, with the opposite ends of these two capacitors attached to ground. The PI filter is able to substantially attenuate signals, allowing the signal passed from the base 200 of the injector 105 to the injector head 130 to be substantially free from RF interference. The injector head 130 may or may not need to be shielded further from such interference. The PI filter may exist as part of the interface card.

The electronics in the injector head 130 preferably consist of “passive” electronics that contain no active digital components. Therefore, there is little or no RF interference radiating from these injector head components. Therefore, any substantial noise in the injector head 130 would most likely come from the base 200 on the injector assembly, and this noise should be attenuated at the base.

The injector head 130 also preferably has a sensor 290 that automatically senses which kind of syringe 250 is inserted into the sensor head 130. Because prior versions of this head sensor 290 created RF noise that may interfere with the MRI image, an improved injector head preferably includes a syringe sensor 290 that is optical.

The base assembly 200 is preferably also designed to have a low center of gravity (to reduce the likelihood of tipping) and has a plurality of legs 292 extending outwards therefrom which supply both support for the base as well as a measured distance from which the base assembly 200 must be positioned from other physical features, such as the MRI equipment or the patient. The base assembly 200 may also have a plurality of casters 294 attached to enable the injection assembly 105 to be moved around the MRI chamber 115. However, because the EMI magnetic field will typically attract the injector assembly 105, the casters 294 are preferably not free-rolling to decrease the likelihood that the MRI magnet may displace the injection assembly. A roll-resistant wheel or caster 294 is preferred.

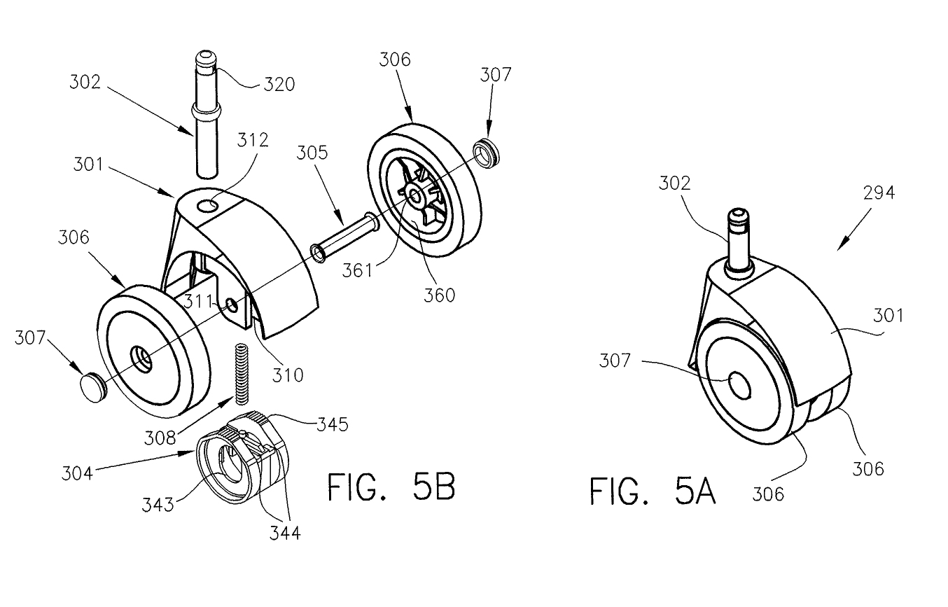

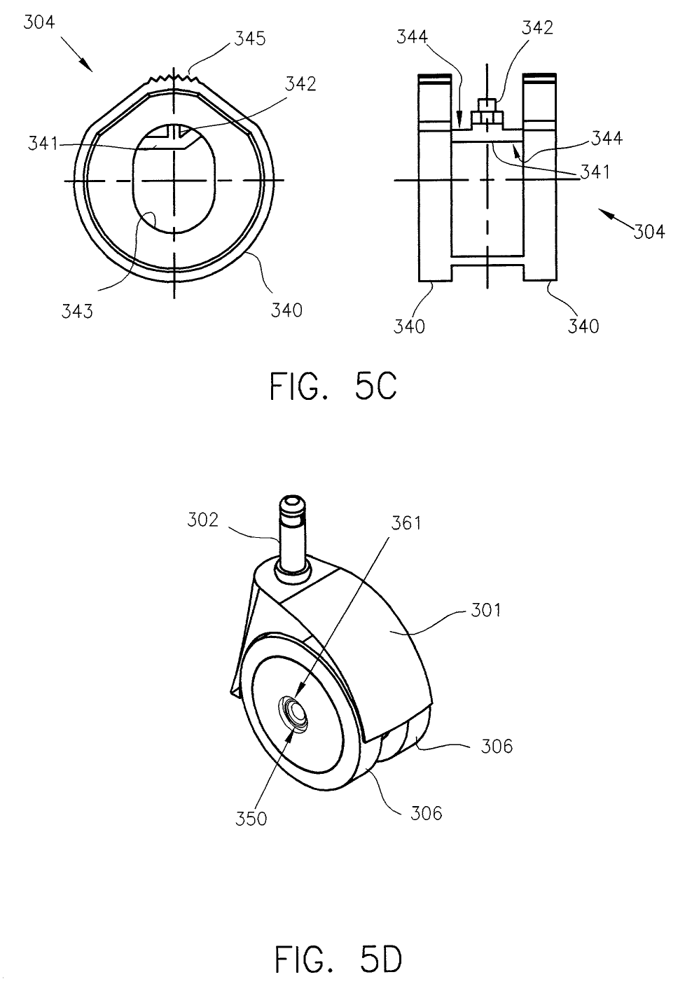

In an ideal embodiment illustrated in FIGS. 5A-5D, a twin-wheel roll-resistant non-magnetic swivel caster 294 is preferred. In particular, the caster 294 of the present invention has a novel roll-resistant design, which constitutes an advance over the free rolling caster assemblies manufactured by companies such as The Jilson Group, Caster Products Division, of Lodi, N.J. It includes a hooded housing 301, a stem 302, a compression spring 303, a dual disc friction pad element 304, an axle 305, two wheels 306 and, optionally, two end caps 307. The hooded housing 301, the dual disc pad element 304 and the rims of wheels 306 may be composed of nylon or other suitable material, and the tires of wheels 306 of nylon, thermoplastic rubber or polyurethane. The stem 302 and the axle 305 are preferably made of brass, stainless steel or other materials appropriate to an MRI environment.

As best shown in FIG. 5A, the hooded housing 301 has a two pronged fork 310 projecting downwardly from the underside of its hood. The two prongs are disposed in parallel with each other and generally aligned in the same direction as the hood. The prongs each define a bore 311, with the bores 311 being axially aligned to accommodate the axle 305 as described further below. Between the prongs lies a receptacle or upper spring stop (not shown) for accommodating the upper end of compression spring 303 upon assembly of the caster 294. The hooded housing 301 with the aforementioned features is preferably molded as a single piece.

The friction pad element 304 is also preferably molded as a single piece. It has two discs 340 disposed in parallel but interconnected by a connective member 341. At the top and center of connective member 341 is formed a nub or lower spring stop 342 for accommodating the lower end of compression spring 303 upon assembly. Each of the discs 340 defines at its center a bore 343 of predetermined shape to allow the axle 305 to pass therethrough. In between the two discs 340 on opposite sides of central nub 342, there are two horizontally disposed slots 344 defined in the connective member 341. These are designed to accommodate the prongs of fork 310 upon assembly of the caster 294.

During assembly, the axle 305 is inserted up to one end thereof in the hub 361 of one wheel 306. The compression spring 303 is then inserted by its upper end into the receptacle (not shown) that lies between the prongs of fork 310 of housing 301. The dual disc pad element 304 is then installed onto the fork 310 of hooded housing 301. More specifically, the prongs of fork 310 are inserted into the corresponding slots 344 of connective member 341. As the prongs are moved into slots 344., the central nub 342 at the top of connective member 341 shall be fitted concentrically within the lower end of compression spring 303. At this point in the assembly process, each of the two prong bores 311 of fork 310 will typically be visible through and partially aligned with its corresponding bore 343 in element 304.

The wheel 306 into which the axle 305 has already been inserted is then ready to be installed into the overall assembly. Specifically, the free end of axle 305 is inserted through the partially aligned bores 343 and 311 of element 304 and fork 310, respectively. In order to complete this part of the assembly process, however, it will be necessary to push the disc pad element 304 against the compressive force of spring 303 upward towards the underside of housing 301. This allows not only the axle 305 to be inserted through the bores 343 and 311 but also one disc pad 340 of element 304 to be inserted within the drum 360 of one wheel 306. It should be noted that a snug fit of the prongs of fork 310 within the slots 344 of element 304 is desired to avoid freedom of movement in directions other than that required herein.

The other wheel 306 can then be installed onto the free end of axle 305. To do so, however, it will again be necessary to push the disc pad element 304 against the compressive force of spring 303 upward towards the underside of housing 301. This allows not only the hub 361 to be fitted onto the free end of axle 305 but also the drum 360 of other wheel 306 to accommodate the other disc pad 340 of dual disc pad element 304. Once both wheels 306 are installed, a flange 350 can be formed on each end of axle 305 for the purpose securing the wheels 306 to axle 305. An end cap 307 may be fitted into outer side of the hub 361 of each wheel 306 to protect the wheel/axle assemblies from dust, dirt and other contaminants.

Regarding attachment of caster 294 to the injection apparatus 105 for which it is preferably intended, the lower part of stem 302 mounts within a corresponding bore 312 defined in the top of hooded housing 301. The upper part of stem 302 should have a groove and associated grip ring 320 or other suitable attachment means that enables the stem 302 to snap into a corresponding slotted bore (not shown) in a leg 292 of injection apparatus 105. From FIGS. 5A, 5B and 5D, it can be seen that the bore 312 is preferably axially offset from the prongs of fork 310. This allows the assembly to act as a caster, as the lower part of stem 302 is preferably free to swivel in bore 312 of hooded housing 301. Once assembled in the aforementioned manner, one or more casters 294 may be attached to each leg 292 of injection apparatus 105.

Due to the constant force with which compression spring 303 pushes the discs 340 of friction pad element 304 against the drums 360, the wheels 306 are resistant to rolling. For this reason, the spring constant of compression spring 303 should be selected with regard to the MRI environment for which the invention is preferably intended. The spring constant, however, should not be so great as to prevent rolling of the wheels 306 when the injection apparatus 105, to which the casters 294 are attached, is being moved by medical or other appropriate personnel.

From the foregoing, it should be apparent that, in this preferred embodiment of roll-resistant non-magnetic caster 294 described herein, the ridges 345 on the top surface of each disc 340 of dual disc friction pad element 304 need not be present.

Nothing in the above description is meant to limit the present invention to any specific materials, geometry, or orientation of parts. Many part/orientation substitutions are contemplated within the scope of the present invention. The embodiments described herein were presented by way of example only and should not be used to limit the scope of the invention.

Although the invention has been described in terms of particular embodiments in an application, one of ordinary skill in the art, in light of the teachings herein, can generate additional embodiments and modifications without departing from the spirit of, or exceeding the scope of, the claimed invention. Accordingly, it is understood that the drawings and the descriptions herein are proffered by way of example only to facilitate comprehension of the invention and should not be construed to limit the scope thereof.

We claim:

1. An injection system for use with a magnetic resonance imaging (MRI) system, said MRI system having a room and a window therewith shielded from electromagnetic interference, said injection system comprising:
(a) a system controller external to said room;
(b) an injection apparatus within said room, said injection apparatus including a base assembly, a head assembly and a rigid tubular casing for supporting said head assembly above said base assembly and for housing a flexible drive shaft, said base assembly including a motor control circuitry and an electric drive motor controlled thereby operably linked to one end of said flexible drive shaft, said head assembly including an injector and a drive mechanism therefor operably linked to an other end of said flexible drive shaft; and
(c) a communications control link, substantially non-reactive with a magnetic field produced by said MRI system, between said system controller and said injection apparatus thereby enabling said system controller to operably control said base assembly and said head assembly therewith for controlling the injection of fluid into a patient from a syringe mounted to said injector during a magnetic resonance imaging procedure.

2. The injection system of claim 1 wherein said base assembly has an enclosure configured to act a Faraday cage to minimize electromagnetic interference within said room.

3. The injection system of claim 2 wherein said head assembly has an enclosure configured to act a Faraday cage to minimize electromagnetic interference within said room.

4. The injection system of claim 3 wherein a communications conduit between said head and said base assemblies is electromagnetically shielded thereby extending Faraday cage isolation to all electromagnetic components connected to said base assembly.

5. The injection system of claim 4 wherein said injection apparatus further comprises a hand switch for providing local control of said injection apparatus, said hand switch communicating with and operable through said base assembly.

6. The injection system of claim 2 wherein said motor control circuitry comprises a plurality of circuit boards, with each of said circuit boards having an orientation in said base assembly and an architecture of circuit components and traces thereon configured to minimize electromagnetic emissions therefrom.

7. The injection system of claim 6 wherein said circuit boards include a CPU/servo card, a power drive card and an interface card.

8. The injection system of claim 2 wherein said electric drive motor is oriented within said base assembly to at least one of minimize torque needed to drive said flexible drive shaft and limit interaction with the magnetic field produced by said MRI system.

9. The injection system of claim 1 wherein said electric drive motor is a three-phase brushless type motor.

10. The injection system of claim 1 wherein said injection apparatus further comprises a hand switch for providing local control of said injection apparatus.

11. The injection system of claim 10 wherein said hand switch is operable through and connected to said base assembly via an electromagnetically shielded cable.

12. The injection system of claim 1 wherein said head assembly further comprises a sensor for automatically detecting a type of syringe mounted to said injector.

13. The injection system of claim 12 wherein said sensor is an optical sensor.

14. The injection system of claim 1 wherein said injection apparatus further comprises a rechargeable battery for providing power to said injection apparatus.

15. The injection system of claim 14 wherein said rechargeable battery is removably installed into said base assembly.

16. The injection system of claim 1 wherein said communications control link includes means for transmitting and receiving electromagnetic signals through said window.

17. The injection system of claim 16 wherein said electromagnetic signals are in the visible frequency part of the electromagnetic spectrum.

18. The injection system of claim 16 wherein said electromagnetic signals are in the infrared frequency part of the electromagnetic spectrum.

19. The injection system of claim 16 wherein said electromagnetic signals are in the radio frequency part of the electromagnetic spectrum.

20. The injection system of claim 1 wherein said communications control link includes means for transmitting and receiving electromagnetic signals.

21. The injection system of claim 1 wherein said communications control link comprises a fiber optic cable.

22. An injection system for use with a magnetic resonance imaging (MRI) system, said MRI system having a room and a window therewith shielded from electromagnetic interference, said injection system comprising:
(a) a system controller external to said room;
(b) an injection apparatus within said room, said injection apparatus including a head assembly, a base assembly and a rigid tubular casing for supporting said head assembly above said base assembly and for housing two flexible drive shafts, said head assembly including (i) an injector adaptable to accommodate two syringes mountable thereon and (ii) a drive mechanism for each of said flexible drive shafts such that each said drive mechanism is linked to one end of said flexible drive shaft corresponding thereto for operating one of said syringes corresponding thereto, said base assembly operably engaged with each of said flexible drive shafts at other ends thereof with which to drive said drive mechanisms and operate said syringes independently therewith in response to commands from said system controller;
(c) a communications control link between said system controller and said injection apparatus thereby enabling said system controller to operably control said base assembly and said head assembly therewith for controlling the injection of fluid(s) into a patient from at least one of said syringes during a magnetic resonance imaging procedure.

23. The injection system of claim 22 wherein said base assembly has an enclosure configured to act a Faraday cage to minimize electromagnetic interference within said room.

24. The injection system of claim 23 wherein said head assembly has an enclosure configured to act a Faraday cage to minimize electromagnetic interference within said room.

25. The injection system of claim 24 wherein a communications conduit between said head and said base assemblies is electromagnetically shielded thereby extending Faraday cage isolation to all electromagnetic components connected to said base assembly.

26. The injection system of claim 25 wherein said injection apparatus further comprises a hand switch for providing local control of said injection apparatus, said hand switch communicating with said base assembly through said electromagnetically shield conduit.

27. The injection system of claim 23 wherein said base assembly includes:
(a) two electric drive motors, with each of said electric drive motors engaged with one of said flexible drive shafts corresponding thereto with which to drive one of said drive mechanisms corresponding thereto, said electric drive motors being oriented within said base assembly to at least one of minimize torque needed to drive said flexible drive shafts and limit interaction with the magnetic field produced by said MRI system; and
(b) a motor control circuitry comprising a plurality of circuit boards, with each of said circuit boards having an orientation in said base assembly and an architecture of circuit components and traces thereon configured to minimize electromagnetic emissions therefrom.

28. The injection system of claim 27 wherein said circuit boards include a CPU/servo card, a power drive card and an interface card.

29. The injection system of claim 27 wherein said electric drive motors are three-phase brushless type motors.

30. The injection system of claim 22 wherein said injection apparatus further comprises a hand switch for providing local control of said injection apparatus.

31. The injection system of claim 30 wherein said hand switch is operable through and connected to said base assembly via an electromagnetically shielded conduit.

32. The injection system of claim 22 wherein said head assembly further comprises a sensor for automatically detecting a type of syringe mounted to said injector.

33. The injection system of claim 32 wherein said sensor is an optical sensor.

34. The injection system of claim 22 wherein said injection apparatus further comprises a rechargeable battery for providing power to said injection apparatus.

35. The injection system of claim 34 wherein said rechargeable battery is removably installed into said base assembly.

36. The injection system of claim 22 wherein said communications control link includes means for transmitting and receiving electromagnetic signals through said window.

37. The injection system of claim 36 wherein said electromagnetic signals are in the visible frequency part of the electromagnetic spectrum.

38. The injection system of claim 36 wherein said electromagnetic signals are in the infrared frequency part of the electromagnetic spectrum.

39. The injection system of claim 36 wherein said electromagnetic signals are in the radio frequency part of the electromagnetic spectrum.

40. The injection system of claim 22 wherein said communications control link includes means for transmitting and receiving electromagnetic signals.

41. The injection system of claim 22 wherein said communications control link comprises a fiber optic cable.

42. A roll-resistant caster assembly for an injection apparatus of the type used with a magnetic resonance imaging (MRI) system, said roll-resistant caster assembly comprising:
(a) an axle;
(b) two wheels each of which defining a hub for attachment to one end of said axle, each of said wheels also defining a drum on an interior periphery thereof,
(c) a housing having a fork portion characterized by two prongs projecting downwardly therefrom, each of said prongs defining an axle bore therethrough with said axle bores being axially aligned to accommodate said axle for support of said housing thereon, said housing also having an upper spring stop disposed between said prongs in an upper part of said fork portion;
(d) a dual disc pad element having two discs and a connective member interconnecting said discs so that said discs are disposed in parallel, said discs each defining a central bore to accommodate compressive movement of said dual disc pad element relative to said axle situated therein and said fork portion therewith, said connective member including a lower spring stop at a top and a center thereof, said connective member also defining two slots each of which on opposite sides of said lower spring stop and each adapted to accommodate one of said prongs of said fork portion of said housing; and
(e) a spring disposed compressively between said upper and said lower springs stops such that said spring normally biases said discs of said dual disc pad element against said drums of said wheels thereby rendering said wheels of said caster assembly resistant to rolling.

43. The roll-resistant caster assembly of claim 42 wherein a spring constant of said spring is selected so that said wheels shall be able to roll when said injection apparatus to which said caster assembly is attached is moved by human intervention.

44. The roll-resistant caster assembly of claim 42 wherein a spring constant of said spring should be selected with regard to an MRI environment in which said caster assembly may be used.

45. The roll-resistant caster assembly of claim 42 further comprising a stem having a lower part and an upper part such that:
(a) said lower part of said stem is for mounting and rotating within a stem bore defined within a top of said housing, said stem bore being axially offset from said prongs of said fork portion thereby allowing said caster assembly to swivel about said stem; and
(b) said upper part of said stem having an attachment means that enables said stem to be secured into a corresponding bore in a leg of said injection apparatus.

46. The roll-resistant caster assembly of claim 42 wherein said housing includes a hood that covers an upper portion of an assembly comprising said wheels and said axle.

47. The roll-resistant caster assembly of claim 42 wherein said axle has flanges formed on each end thereof for the purpose securing said wheels thereon.

48. The roll-resistant caster assembly of claim 42 further comprising an end cap fitted into each of said hubs of said wheels on an outer side thereof.

49. The roll-resistant caster assembly of claim 42 being made of materials appropriate for an MRI environment.

50. The roll-resistant caster assembly of claim 49 wherein said housing, said dual disc pad element and said wheels are each made of at least one of nylon, thermoplastic rubber and polyurethane.

51. The roll-resistant caster assembly of claim 49 wherein said stem and said axle are each made of at least one of brass and stainless steel.
